(19) United States
(12) Patent Application Publication
(10) Pub. No.: US 2009/0286837 A1
(43) Pub. Date: Nov. 19, 2009

(54) Oxadiazole compounds as urokinase inhibitors

(76) Inventors: Stefan Sperl, Wien (AT); Jana Zimmermann, Wien (AT); Klaus Koch, Mering (DE)

Correspondence Address: MCDERMOTT WILL & EMERY LLP, 600 13TH STREET, N.W., WASHINGTON, DC, 20005-3096, US

(21) Appl. No.: 11/991,268

(22) PCT Filed: Aug. 29, 2006
(86) PCT No.: PCT/EP2006/008451

§ 371 Date: Feb. 29, 2008

(30) Foreign Application Priority Data
Aug. 29, 2005 (EP) 05018732.7

(51) Int. Cl. A61K 31/225; A61K 31/4245; C07C 69/34; C07D 271/07; A61P 35/04

(52) U.S. Cl. 514/364; 514/548; 560/194; 548/133

(57) ABSTRACT

The present invention relates to novel compounds which inhibit urokinase-plasminogen activator (uPA), have a high bioavailability and can be administered orally, and to their use as therapeutic active substances for the treatment of disorders associated with urokinase and/or urokinase receptor, for example tumours and metastasis. The invention relates in particular to compounds containing oxadiazole groups.

The present invention relates to novel compounds for inhibiting the urokinase plasminogen activator (uPA), which have high bioavailability and oral administerability, and also to the use thereof as therapeutic active compounds for the treatment of urokinase- or/and urokinase receptor-associated disorders such as, for example, tumors and metastasizing. The invention relates in particular to compounds containing oxadiazole groups.

The plasminogen activator of the urokinase type (uPA) plays a key part in tumor invasion and the formation of metastases (Schmitt et al., J. Obst. Gyn. 21 (1995), 151-165). uPA is expressed in many different types of tumor cells (Kwaan, Cancer Metastasis Rev. 11 (1992), 291-311) and binds to the tumor-associated uPA receptor (uPAR) where activation of plasminogen to plasmin occurs. Plasmin is capable of degrading various components of the extracellular matrix (ECM), such as fibronectin, laminin and type IV collagen. It also activates some other ECM-degrading enzymes, in particular matrix metalloproteinases. Large amounts of tumor-associated uPA correlate with a higher risk of metastasizing for cancer patients (Harbeck et al., Cancer Research 62 (2002), 4617-4622). Inhibition of the proteolytic activity of uPA is therefore a good starting point for an antimetastatic therapy.

Some active and selective urokinase inhibitors have been described previously. Thus, EP 1 098 651 discloses benzamidine-type uPA inhibitors, and WO 01/96286 and WO 02/14349 disclose arylguanidine-type uPA inhibitors. A common feature of these synthetic inhibitors is a basic radical consisting of an amidino or/and guanidino group.

However, the known urokinase inhibitors have the disadvantage of being absorbed poorly when applied orally and thus can exert only a low pharmacological action in the body with this type of administration. Pharmaceutical preparations are therefore administered to the patient intravenously usually once, but up to twice weekly over a period of several hours. This puts a great strain on the patient, since this requires considerable time and frequent hospital visits and demands a high level of cooperation of the patient.

Moreover, intravenous administration carries the risk of infections and, especially in the case of para-vasally escaping infusate, severe local irritations up to tissue necroses may occur, which require time-consuming subsequent treatments and monitoring.

Intramuscular and subcutaneous routes of administration also do not offer any advantages, since here frequently severe pain at the injection sites and also irritations up to tissue necroses may occur, which likewise require a time-consuming after-treatment.

As discussed above, the amidine- and guanidine-containing urokinase inhibitors exhibit only low pharmacological action when applied orally. A precondition for the therapeutic effect of an active compound is the bioavailability of the latter. Oral administration requires absorption from the gastro-intestinal tract. An important mechanism of this kind of membrane penetration is passive diffusion (Gangvar S. et al., DDT (1997) 148-155). The lipophilicity of an active compound was assumed in some parts of the literature to play an important part in passive diffusion via the membrane barriers of the gastro-intestinal tract. Thus, EP 0 708 640 describes for pentamidines with antihelminthic action a modification of amidine functions to give amidoxime, amidoxime ester and oxadiazole, with preference being given to using the amidoxime esters and oxadiazole as suitable modifications.

On the other hand, however, it was shown that the degree of lipophilicity alone is not sufficient (Hansch et al., J. Am. Chem. Soc. 86 (1964) 1616-1626) and that an increase in the lipophilicity of the compounds is not an appropriate parameter for predicting membrane penetration. Thus, a direct relation between lipo-philicity and membrane permeation was not found (Conradi et al., Pharm. Res. 9 (1992) 435-439).

The increase in lipophilicity may therefore, in individual cases, increase membrane permeation, but not necessarily lead to an increased oral bioavailability. Thus, in the case of argatroban, conversion of the basic radical to the amidoxime as a prodrug results in improved permeability but, in addition, in the loss of activity (Rewinkel, Adang Cur. Pharm. Design 5 (1999) 1043-1075). It is therefore not readily predictable, whether and which modifications can improve membrane penetration of an active compound in the gastro-intestinal tract. It is even less predictable which influence said modifications may have on the pharmaceutical properties of the active compound.

It was an object of the present invention to provide novel medicaments for inhibiting urokinase, whose bioavailability and activity in the organism, in the case of oral administration, is distinctly increased.

According to the invention, this object is achieved by a medicament, which comprises, as an active compound, one or more compounds of the general formula I

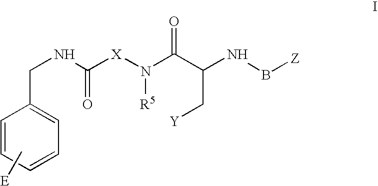

in which
E is a group from

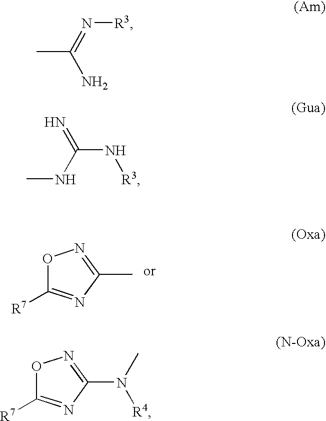

B is —SO₂— or —CO—,
X is —NR¹— or —CHR¹—,
Z is —R⁴, —OR⁴ or —NH—R⁴,
Y is —OR² or —NHR²,
R¹ is in each case independently —H, branched or straight-chain —C₁-C₆-alkyl, —C₂-C₆-alkenyl or —C₂-C₆-alkynyl, unsubstituted or substituted or a cyclic radical,
R² is —H, —R¹, —COR¹, —COOR¹ or —CON(R¹)₂,
R³ is H or —O—R⁸,
R⁸ is —H, —C₁-C₆-alkyl, —C₂-C₆-alkenyl or —C₂-C₆-alkynyl, unsubstituted or substituted, or —COR⁶ or —COOR⁶ or an oligo- or polyalkyleneoxy radical, for example with 2-50 —C₂-C₄-alkyleneoxy, for example ethyleneoxy, groups,
R⁴ is —H, —C₁-C₆-alkyl, —C₂-C₆-alkenyl or —C₂-C₆-alkynyl, unsubstituted or substituted, or a cyclic radical,
R⁵ is —H, —C₁-C₆-alkyl, —C₂-C₆-alkenyl or —C₂-C₆-alkynyl, unsubstituted or substituted, or a cyclic radical,
R⁶ is —H, branched or straight-chain —C₁-C₆-alkyl, C₂-C₆-alkenyl or —C₂-C₆-alkynyl, unsubstituted or substituted, or a cyclic radical,
R⁷ is H, branched or straight-chain, linear, mono-, bi- or polycyclic alkyl, alkenyl, alkynyl, carboxyalkyl, carboxyalkenyl, carboxyalkynyl, aryl, heteroaryl, carboxyaryl, carboxyalkylaryl, carboxyheteroaryl, —(CO)NR¹R⁴ or —COO—R⁴,
   with each cyclic radical being able to carry one or more substituents, for example selected from the group consisting of —C₁-C₃-alkyl, —OR⁶ (e.g. —OH or —C₁-C₃-alkoxy), halogen, in particular Cl, ═O, —NO₂, —CN, —COOR⁶—N(R⁶)₂, —NR⁶COR⁶, —NR⁶CON(R⁶)₂ and —OCOR⁶,
   and it being possible for each alkyl, alkenyl or alkynyl to be straight-chain or branched and to carry one or more substituents, for example selected from the group consisting of halogen (F, Cl, Br, I), —OR⁶, —OCOR⁶, —N(R⁶)₂, —NR⁶COR⁶, COOR⁶, —NR⁶CON(R⁶)₂ or a cyclic radical,
   it not being possible, when Y=OH and E is Am or Gua, for R³ or R⁸ to be H,
   or salts of said compounds and, where appropriate, pharmaceutically customary carriers, diluents or/and excipients.

The medicament is preferably an orally administrable agent. Particular preference is given to using the medicament for inhibiting the urokinase plasminogen activator.

Preference is given to compounds of the general formula II

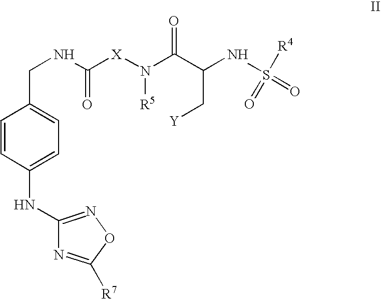

in which
X, Y, R⁴, R⁵ and R⁷ are defined as above, or salts thereof.

The group E is preferably located in the para position of the phenyl ring in compounds I and II. Particular preference is given to compounds of the general formula I, in which E is N-oxa or oxa.

In one embodiment, the compounds of the invention have an oxadiazole group. Surprisingly, such compounds were found to have excellent oral availability.

In a further preferred embodiment, the compounds of the invention have at least one ester, more preferably two ester moieties in positions Y and R³ and/or R⁷. Compounds which have an ester group on the serine residue and/or on group E, in particular on an amidine, a guanidine or an oxadiazole moiety, surprisingly exhibit high efficacy with high oral availability at the same time.

The compounds may be in the form of salts, preferably physiologically compatible acid salts, for example salts of mineral acids, particularly preferably hydro-chlorides, or in the form of salts of suitable organic acids. The compounds may be in the form of optically pure compounds or in the form of mixtures of enantiomers or/and diastereomers.

Cyclic radicals may contain one or more saturated, unsaturated or aromatic rings. Preferred examples of cyclic radicals are cycloalkyl radicals, aryl radicals, alkylaryl radicals, heteroaryl radicals and bicyclic radicals. Particular preference is given to mono- or bicyclic radicals. The cyclic radicals preferably contain from 4 to 30, in particular 5-10, carbon and heteroatoms as ring atoms, and also, where appropriate, one or more substituents, as indicated above. Heterocyclic systems preferably contain one or more O, S or/and N atoms. Preference is given to those bicyclic ring systems having a —CO— radical. Most preferred examples of cyclic radicals are an adamantyl radical or a benzyl radical.

Alkyl, alkenyl and alkynyl groups preferably contain up to 6, in particular up to 4 carbon atoms. R¹ is preferably hydrogen or an unsubstituted or substituted C₁-C₄-alkyl radical, for example —CH₃ or a C₁-C₆-alkyl-aryl radical, so that —CO—X—NR⁵ may be, for example, a glycyl, alanyl, phenylalanyl or homophenylalanyl radical. R¹ in the X moiety is particularly preferably CH₃, and, as a result, X is —CH(CH₃)—.

Particular preference is given to R² being COR¹ and, as a result, Y being an ester group. R¹ in the Y moiety is particularly preferably a branched alkyl radical, in particular tertbutyl.

Particular preference is given to R³ being —O—R⁸, with R⁸ in turn preferably being —COR⁶, as a result of which the R³ moiety preferably comprises an ester. Here too, R⁶ is preferably a branched alkyl radical, in particular tertbutyl. Furthermore, R⁶ is preferably a cyclic radical, in particular adamantyl.

R⁴ is particularly preferably para-chlorobenzyl (Cl—C₆H₅—CH₂—). R⁵ is preferably —H or C₁-C₃-alkyl, in particular CH₃.

Preference is also given to compounds in which the structural element Z is R⁴ which is an alkyl radical having a cyclic substituent, for example an unsubstituted or substituted phenyl radical or a bicyclic radical such as, for example,

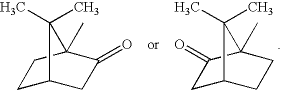

Particular preference is given to those compounds in which R⁴ is a substituted or unsubstituted C₁-C₃-alkyl-aryl radical, for example a benzyl radical, which may be unsubstituted or substituted in the meta or para position with halogen or/and —NO₂, said halogen being selected from the group consisting of F, Cl, Br and I, particularly preferably Cl and Br.

Most preference is given to the compounds given in Table 1 and WX-711 and WX-781 and also salts thereof.

Unless defined otherwise in this text, suitable substituents in each case are halogen, in particular F, Cl, Br or I, C₁-C₄-alkyl, OR⁶, in particular OH, OCOR⁶, COOR⁶, N(R⁶)₂, NR⁶COR₆ or NR⁶CON(R⁶)₂.

The compounds of the invention may be used, where appropriate, together with suitable pharmaceutical excipients or carriers for the preparation of medicaments. Administration is possible here in combination with other active compounds, for example other urokinase inhibitors, such as, for example, antibodies and/or peptides, or else with chemotherapeutics and cytostatics or/and cytostatic active compounds.

Likewise, the compounds of the invention of the general formula I and/or II may also be employed and used like a prodrug.

A prodrug very generally is a pharmaceutically inactive derivative of the corresponding pharmaceutically active substance, which, after oral administration, is converted or transformed spontaneously or enzymatically, whereby said pharmaceutically active substance is released.

The medicaments may be administered to humans and animals topically, rectally or parenterally, for example intravenously, subcutaneously, intramuscularly, intraperitoneally, sublingually, nasally or/and inhalationally, for example in the form of tablets, coated tablets, capsules, pellets, suppositories, solutions, emulsions, suspensions, liposomes, inhalation sprays or transdermal systems such as plasters, and particularly preferably orally, for example as a slow-release formulation.

The compounds of the invention are suitable for controlling diseases associated with pathological over-expression of uPA or/and urokinase plasminogen-activator receptor (uPAR). For example, they are capable of inhibiting in a highly efficient manner the growth or/and the spreading of malignant tumors and metastasizing of tumors. Examples thereof are neoplastic disorders, for example breast cancer, lung cancer, cancer of the bladder, stomach cancer, cervical cancer, ovarian cancer, prostate cancer and soft tissue sarcomas, in particular tumors associated with a high rate of metastasizing. The compounds may be used, where appropriate, together with other tumor agents or with other types of treatment, for example radiation or/and surgical procedures.

The compounds of the invention are furthermore also active for other uPA-associated or/and uPAR-associated disorders. Examples of such disorders are, for example, pulmonary hypertension and/or cardiac disorders (e.g. WO 02/00248), disorders of the stomach and intestine, such as, for example, inflammatory bowel disease, premalignant colon adenomas, inflammatory disorders such as, for example, septic arthritis, osteoarthritis, rheumatoid arthritis, or other disorders such as osteoporosis, cholesteatoma, disorders of the skin and the eyes and also viral or bacterial infections, with reference being made explicitly to the disorders mentioned in EP-A-0 691 350, EP-A-1 182 207 and U.S. Pat. No. 5,712,291.

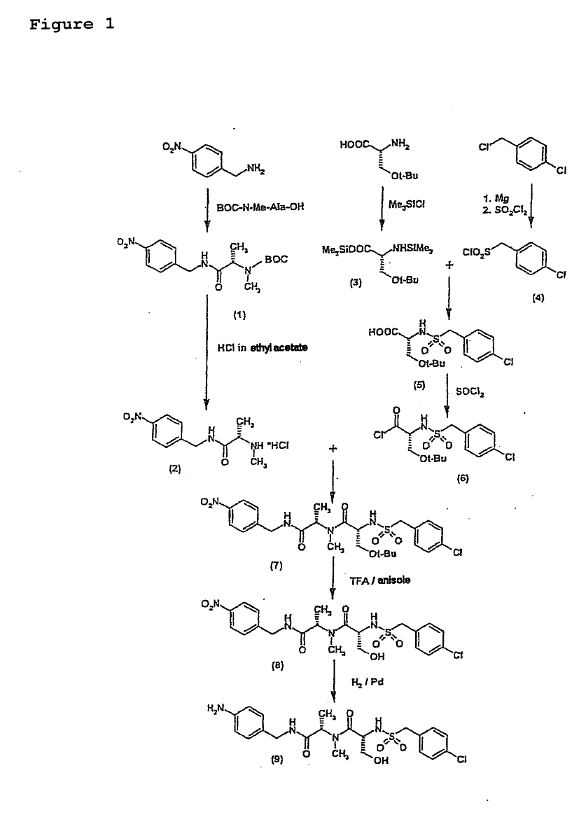

The compounds of the general formula I may be prepared, for example, as in the synthesis flow charts in FIG. 1.

Surprisingly, it was found that the uPA inhibitors of the invention have not only improved bioavailability but also a distinctly improved activity to a primary tumor.

The inventive substances may be used alone or in combination with other physiologically active substances, for example with radiotherapeutics or with cytotoxic or/and cytostatic agents, for example chemotherapeutics, such as, for example, cisplatin, doxorubicin, 5-fluorouracil, taxol derivatives, or/and other chemotherapeutic agents, for example selected from the group consisting of alkylating agents, antimetabolites, antibiotics, epidophyllotoxins and vinca alkaloids. A combination with radiotherapy or/and surgical interventions is also possible.

The invention provides a process for inhibiting urokinase in living organisms, in particular humans, by administering an active amount of at least one compound of the general formula I. The dose to be administered depends on the type and severity of the disorders to be treated. The daily dose, for example, is in the range from 0.01-100 mg/kg active substance.

Finally, the invention relates to novel inhibitors of the urokinase plasminogen activator of the general formula I.

The following figures and the examples are intended to illustrate the invention in more detail.

FIG. 1 depicts a diagram of the WX-770 synthesis and the synthesis of derivatives.

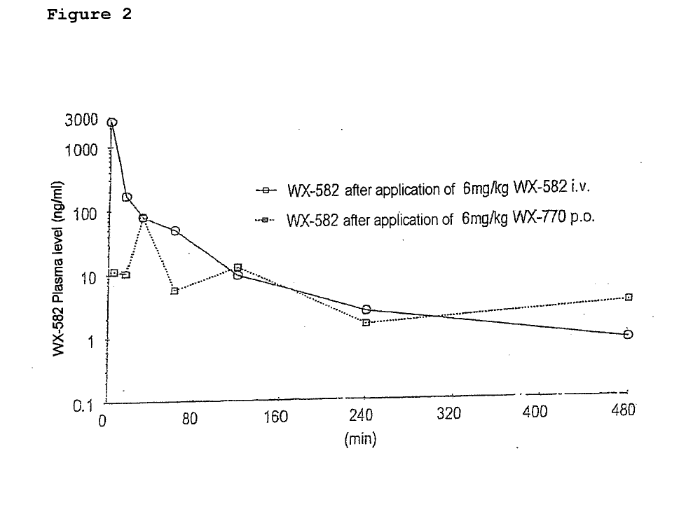

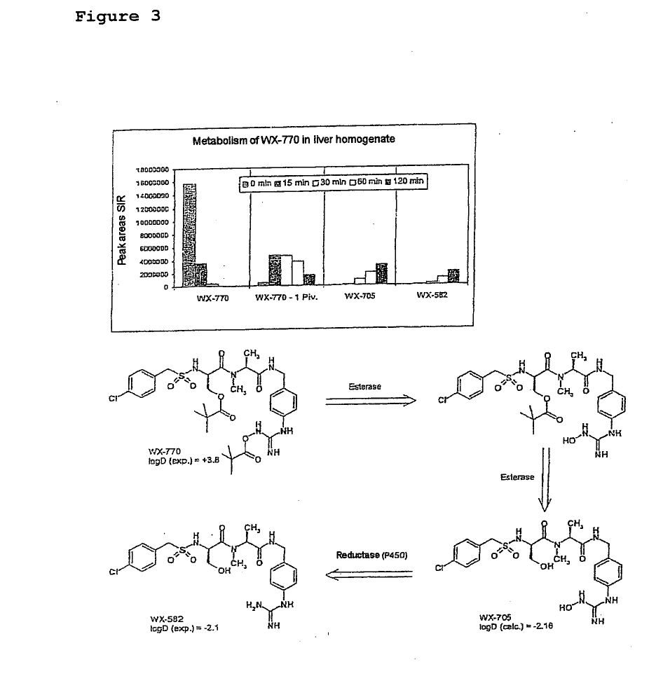

FIG. 2 depicts the oral availability of WX-770 in rats.

FIG. 3 depicts metabolic in vitro activation of WX-770 to give WX-582.

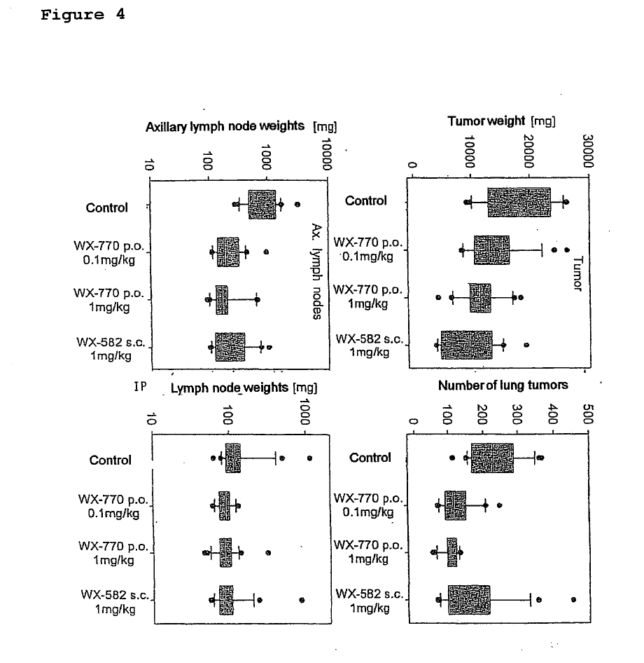

FIG. 4 depicts the efficacy of the compound of the invention, WX-770, in the BN-472 rat tumor model.

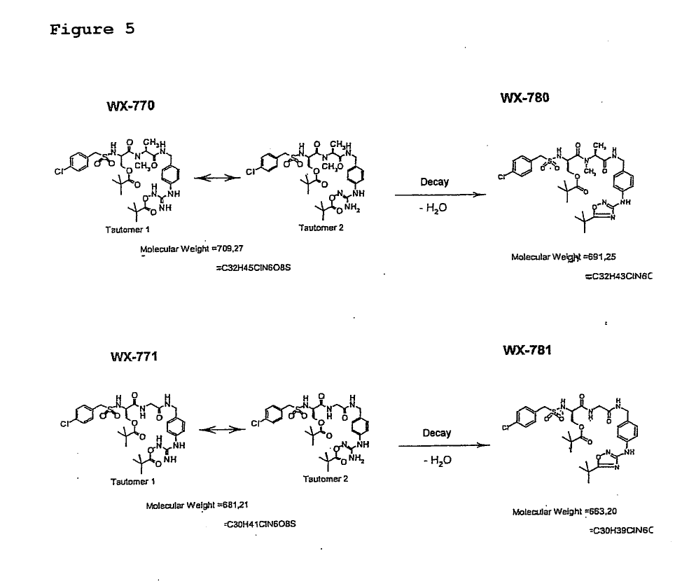

FIG. 5 depicts the rearrangement to give the oxadiazole.

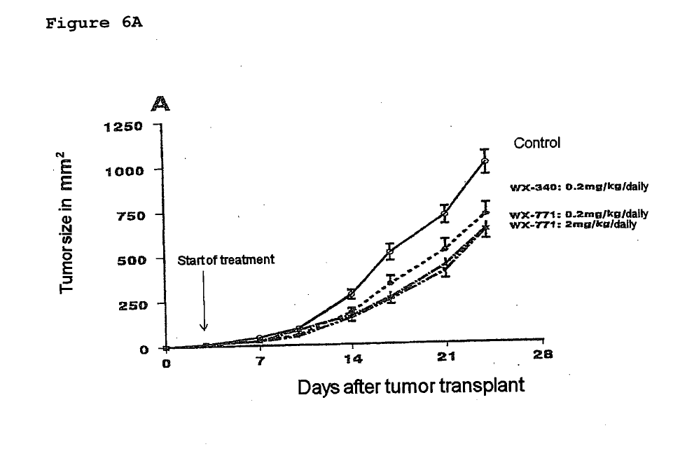

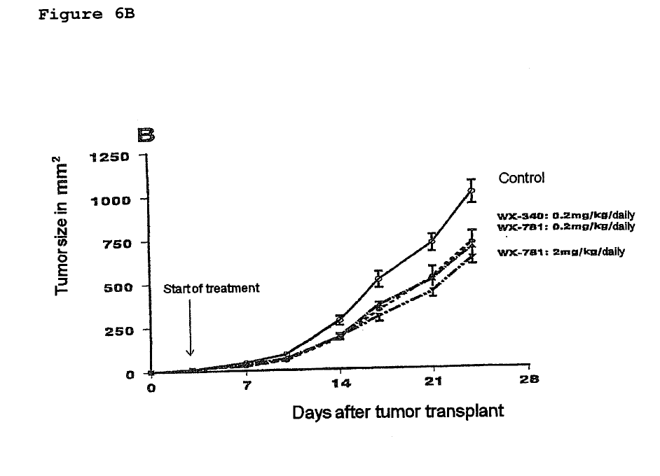

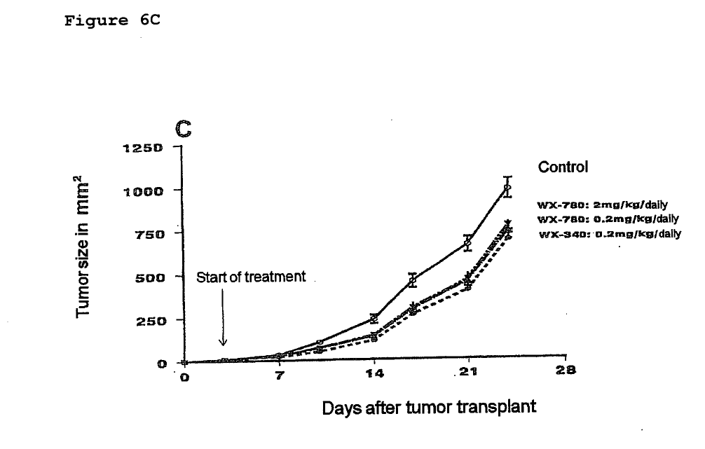

FIG. 6 depicts the tumor growth kinetics for treatment with soya phosphatidylcholine (control), subcutaneous administration of WX-340, and oral administration of WX-771 (FIG. 6A) and WX-781 (FIG. 6B) and for WX-780 (FIG. 6C) in doses of 0.2 mg/kg and 2 mg/kg, respectively.

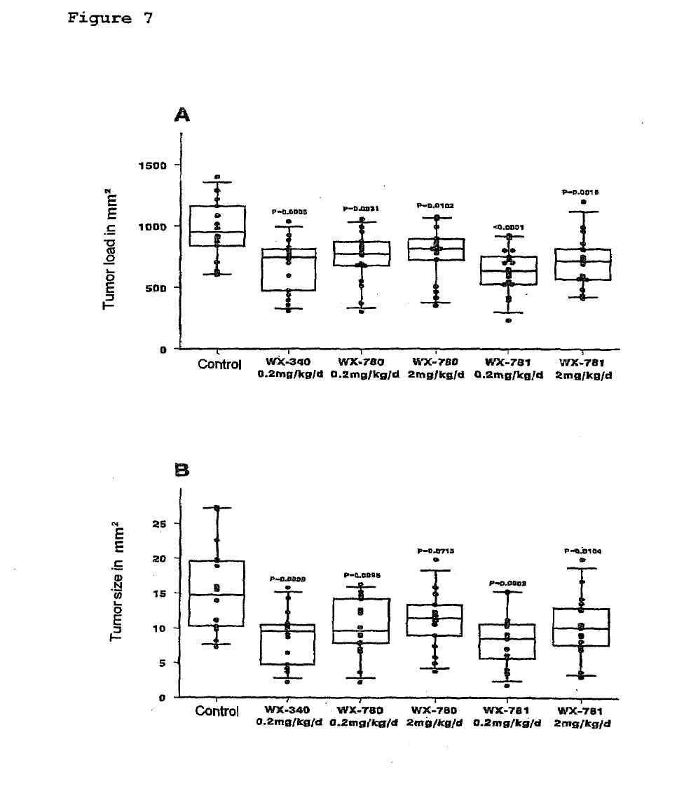

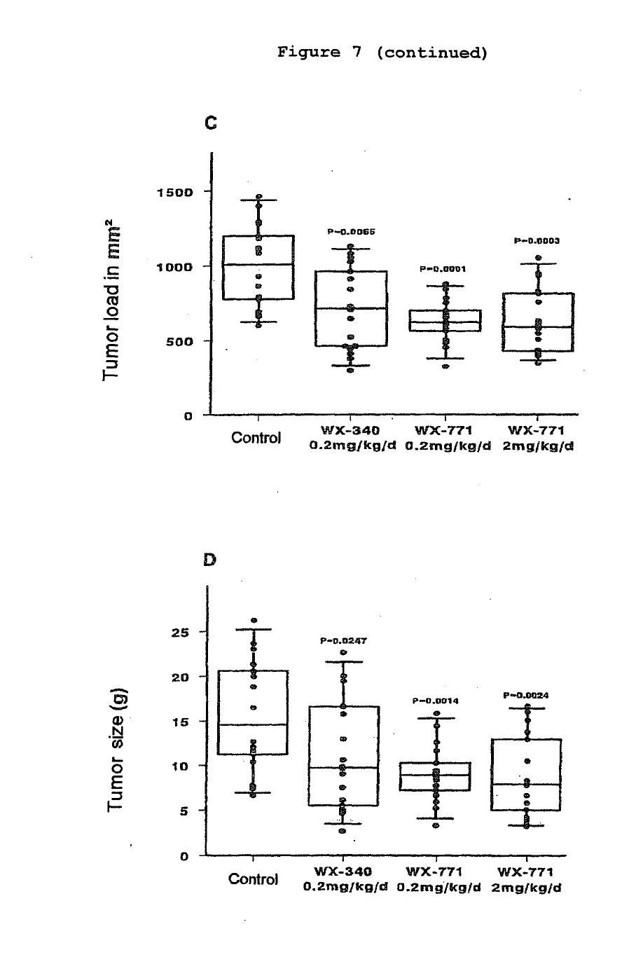

FIG. 7 depicts the effects on the size and weight of the primary tumor.

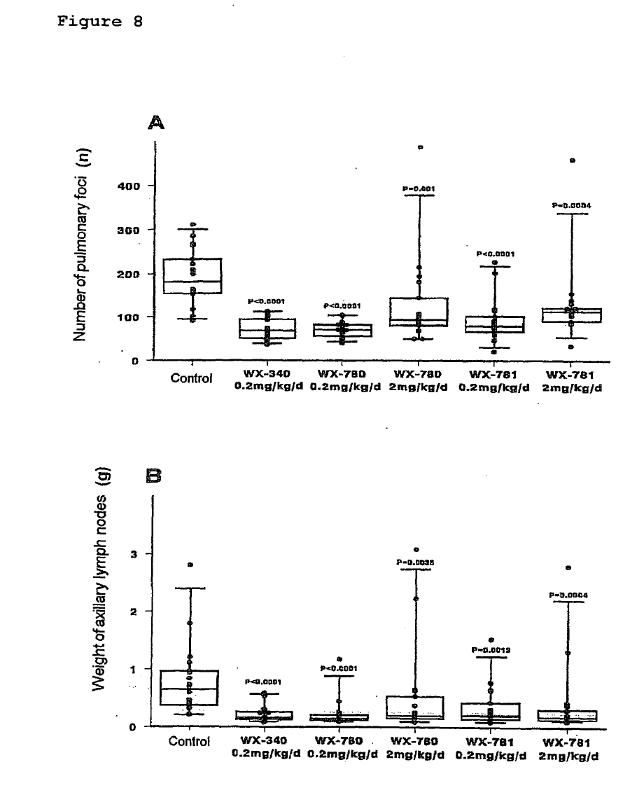

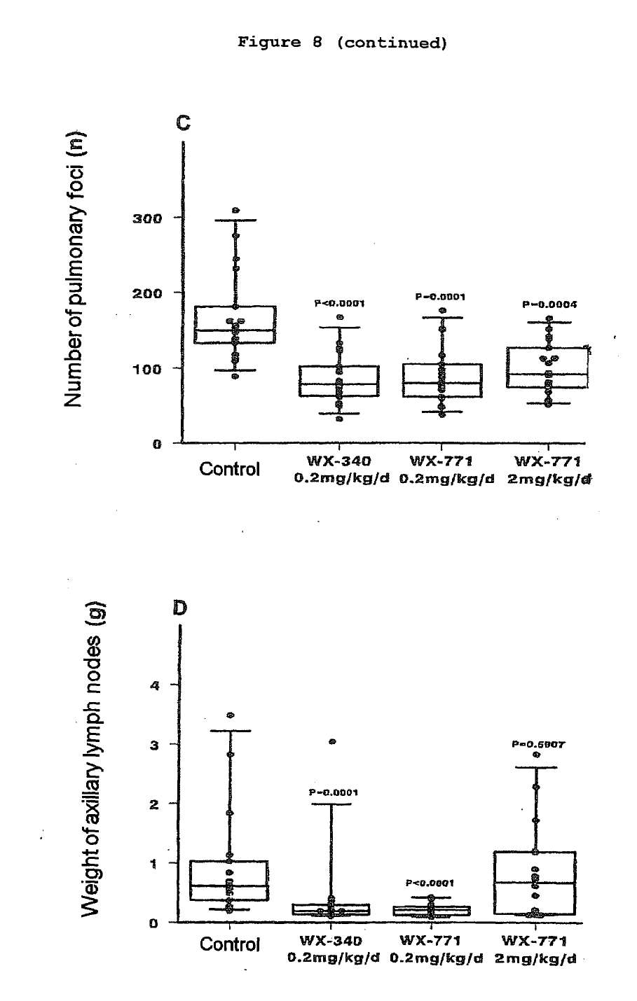

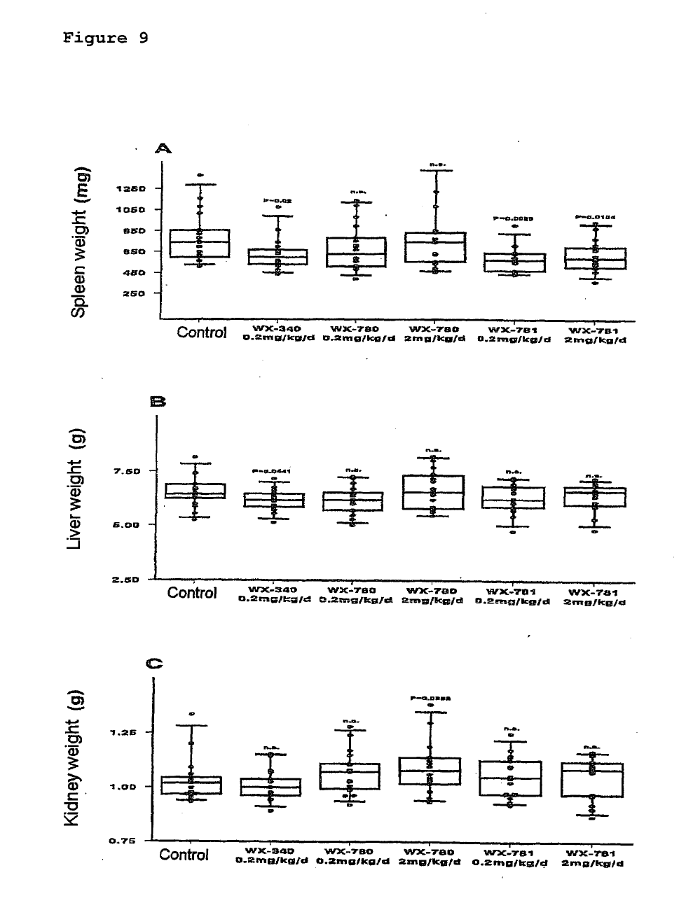

FIG. 8 depicts the anti-metastatic effects.

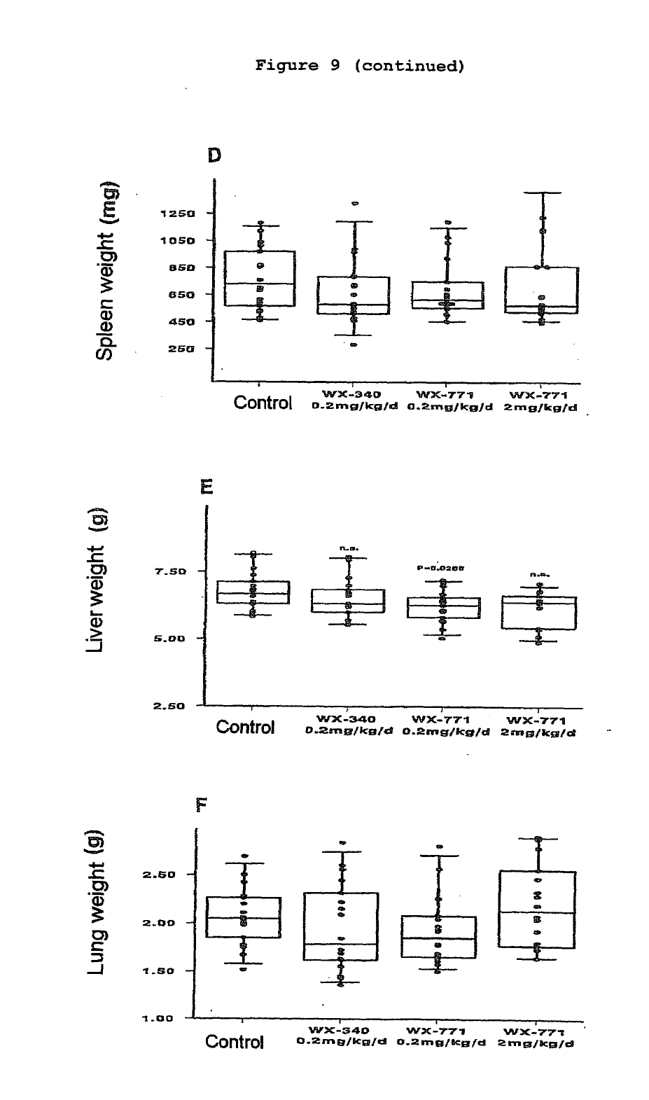

FIG. 9 depicts the effects on organ weights.

### EXAMPLE 1

### Synthesis of WX-770

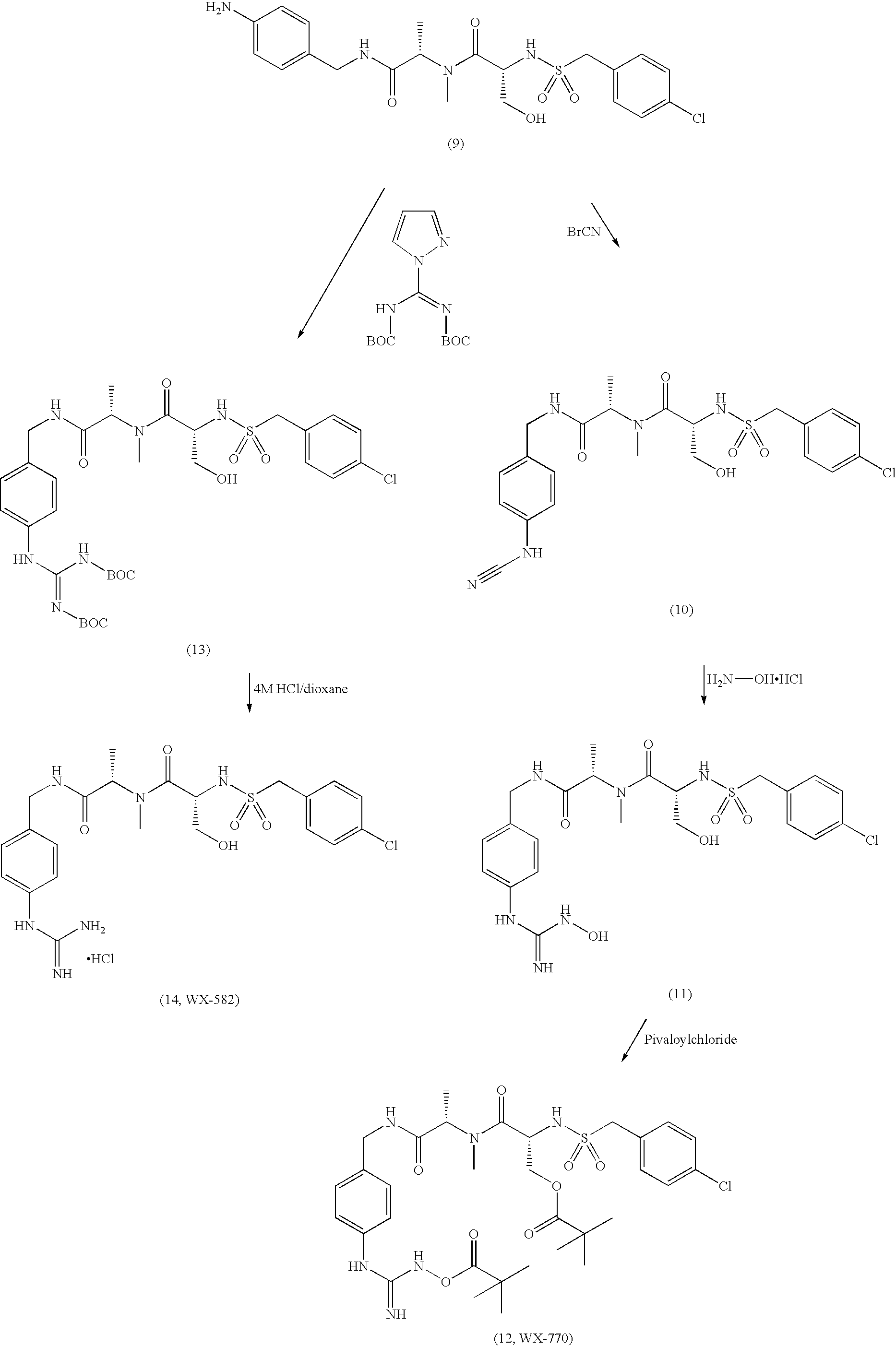

### Boc-N-Me-Ala-4-nitrobenzylamide (1)

N-Hydroxysuccinimide (23.77 g, 206.7 mmol) and N,N′-di-cyclohexylcarbodiimide (33.10 g, 160.7 mmol) were added to a solution of Boc-N-methyl-L-alanine (30.00 g, 147.8 mmol) in dry tetrahydrofuran (980 ml) and stirred at room temperature for 1.5 h. This was followed by adding 4-nitrobenzylamine (22.78 g, 149.9 mmol; obtained from the hydrochloride by treatment with lye) and stirring the mixture overnight (TLC control: CHCl₃/MeOH 5:1). Undissolved components were filtered off, the solids were washed again with THF and the filtrate was concentrated. The residue was purified chromatographically on silica gel with chloroform/methanol (99:1), yielding 1 as a solid (52.8 g).

### N-Me-Ala-4-nitrobenzylamide hydrochloride (2)

A saturated solution of hydrogen chloride in ethyl acetate (1.5 l) was added to a solution of 1 (51.90 g, 153.8 mmol) in ethyl acetate (1.5 l) at −78° C., and the mixture was stirred in a thawing cold bath overnight (TLC control: CHCl₃/MeOH 5:1). Nitrogen was passed through the solution for some time, in order to remove excess hydrogen chloride. This was followed by evaporating to dryness and digesting the residue with ether, and the solid was filtered off and dried (30.48 g, 83%).

### 4-Chlorobenzylsulfonyl chloride (3)

Compound 3 was prepared from 4-chlorobenzyl chloride. From the latter, 4-chlorobenzyl magnesium bromide was first prepared by classical Grignard reaction and then reacted with sulfuryl chloride in a highly exothermic reaction according to: Bhattacharya, S. N.; Eaborn, C.; Walton, D. R. M. J. Chem. Soc. 1968, 1265-1267. However, the product is also commercially available, at least in relatively small amounts.

### (4-Chlorobenzylsulfonyl)-D-Ser(O-tBu)-OH (5)

O-tert-Butyl-D-serine (16.30 g, 101.2 mmol) was suspended under argon in dry dichloromethane (150 ml) and admixed with triethylamine (20.45 g, 202.5 mmol); to this trimethylchlorosilane (21.87 g, 202.5 mmol) was added dropwise with vigorous stirring. A slight warming was observed, and the mixture was subsequently heated to reflux for 1.5 h. The clear solution of 3 was chilled on ice and then admixed with chlorobenzyl-sulfonyl chloride (4, 19.36 g, 86.1 mmol), and the mixture was stirred in the warming cold bath overnight (TLC control: CHCl₃/MeOH 5:1). The reaction mixture was concentrated under reduced pressure and the residue was taken up in ether. The mixture was extracted several times with 5% strength sodium hydrogen carbonate solution, the combined aqueous phases were adjusted to pH 2 with 1 M sulfuric acid, the product was extracted with ethyl acetate, the combined organic phases were dried (MgSO₄) and concentrated (28.65 g, 80%).

### (4-Chlorobenzylsulfonyl)-D-Ser(O-tBu)-Cl (6)

Thionyl chloride (10.55 g, 89.4 mmol) and dry DMF (1.1 ml) were added to a solution of 5 (15.60 g, 44.7 mmol) in dry dichloromethane (425 ml), and the mixture was stirred at room temperature for 3 h (GC control). The solvent was evaporated and the crude acid chloride 6 (17.00 g) was used without further purification.

### (4-Chlorobenzylsulfonyl)-D-Ser(O-tBu)-N-Me-Ala-4-nitro-benzylamide (7)

Diisopropylethylamine (11.54 g, 89.5 mmol) was added to a solution of 2 (12.24 g, 44.7 mmol) in dry dichloro-methane (150 ml), and the mixture was stirred until a clear solution was obtained. A solution of 6 (16.46 g, 44.7 mmol) in dichloromethane (50 ml) was then added dropwise with cooling on ice, and the mixture was stirred at room temperature for 2 h. (TLC control: CHCl₃/MeOH 9:1). The organic solution was washed with 5% strength potassium hydrogen sulfate solution and 5% strength sodium hydrogen carbonate solution, dried (MgSO₄) and concentrated. The residue was chromatographed on silica gel with pentane/ethyl acetate (3:2), yielding 7 as colorless solid (14.73 g, 58%).

### (4-Chlorobenzylsulfonyl)-D-Ser-N-Me-Ala-4-nitrobenzyl-amide (8)

Thioanisole (3.20 g, 25.8 mmol) was added to a solution of 7 (14.70 g, 25.8 mmol) in trifluoroacetic acid (260 ml), and the solution was stirred at room temperature overnight (TLC control: CHCl₃/MeOH 5:1). The reaction mixture was concentrated under reduced pressure and high vacuum. The crude product was purified by column chromatography on silica gel with CHCl₃→CHCl₃/MeOH (5:1), (yield: 10.37 g, 78%).

### (4-Chlorobenzylsulfonyl)-D-Ser-N-Me-Ala-4-aminobenzyl-amide hydrochloride (9 HCl)

10% palladium on activated carbon (wet, contains 50% water; 0.50 g) were added to a solution of 8 (5.00 g, 9.8 mmol) in methanol (50 ml) and hydrogenated overnight (hydrogen balloon). After 3 h, catalyst (0.05 g) was added again (TLC control: CHCl₃/MeOH 5:1). The catalyst was filtered off then washed with methanol, and the filtrate was evaporated to dryness. The free amine 9 (3.86 g, 7.9 mmol) was cooled on ice and suspended in 4 M hydrogen chloride in dioxane (7.8 ml) and then admixed with methanol (10 ml), until a clear solution was obtained. After 30 min of cooling on ice, another 4 M HCl in dioxane (7.8 ml) were added, and the mixture was stirred at 0° C. for another hour. The reaction solution was concentrated and the solid was dried under reduced pressure (4.19 g, 82%).

### (4-Chlorobenzylsulfonyl)-D-Ser-N-Me-Ala-4-(N-cyan-amino)benzylamide (10)

Anhydrous sodium acetate, heated under reduced pressure (1.53 g, 19.1 mml), was added to a solution of 9 (HCl-salt!!!) (3.96 g, 7.6 mmol) in dry ethanol (60 ml). The suspension was admixed with a solution of cyanogen bromide (0.88 g, 8.4 mmol) in dry ethanol (30 ml) and stirred at room temperature overnight. The reaction mixture was cooled in an ice bath, undissolved material was removed by suction, the residue was washed with a little ethanol and the filtrate was concentrated. The residue was purified chromatographically on silica gel with chloroform/methanol (9:1) (1.90 g, 49%).

### (4-Chlorobenzylsulfonyl)-D-Ser-N-Me-Ala-4-(N-hydroxy-guanidino)benzylamide (11)

A solution of 12 (1.90 g, 3.7 mmol) in dry ethanol (38 ml) was cooled on ice and admixed with hydroxylamine hydrochloride (0.26 g, 3.7 mmol) and ethyldiisopropylamine (0.48 g, 3.7 mmol) and stirred at room temperature overnight. On the next day, another 0.2 molar equivalents of hydroxylamine hydrochloride and ethyldiisopropylamine were added. After 24 h, the reaction mixture was concentrated and the residue was chromatographed on silica gel with chloroform/methanol (9:1→5:1) containing 1% acetic acid. The product fractions were concentrated and last remnants of acetic acid were removed by codistillation with toluene. The residue was suspended in a mixture of ether and tetrahydrofuran and stirred. The solid was removed by suction and dried under reduced pressure (1.0 g, 50%).

### (4-Chlorobenzylsulfonyl)-D-Ser(CO-t-Bu)-N-Me-Ala-4-(N-tert-butylcarbonyloxyguanidino)benzylamide (12, WX-770)

A solution of 200 mg of 11 (0.37 mmol) in 2 ml of NMP and 2 ml of pivalic acid was admixed with 5 eq. of pivalic acid chloride (228 μl; 1.85 mmol) and stirred at RT over the weekend. This resulted in a mixture of approx. 60% product, 20% reactants and 20% of the in each case mono-pivalic ester-substituted reactants. After the addition of another 5 eq. of pivalic acid chloride, the reaction was stopped after a further 2 days. According to HPLC, the product had formed with high purity. The reaction mixture was diluted with 20 ml of ethyl acetate and extracted 3× with 20 ml each of 5% NaHCO₃ and 1× with concentrated NaCl solution. The organic phase was dried over Na₂SO₄ and the solvent was evaporated on a rotary evaporator. The resulting oil was dissolved in 5 ml of dichloromethane and slowly added dropwise with stirring, to 50 ml of diisopropyl ether. The flocculent precipitate was left in a refrigerator to crystallize further, then removed by centrifugation and dried under high vacuum. Yield: 115 mg (0.16 mmol; 44%).

### Anti-Tumor Efficacy and Anti-Metastatic Efficacy of WX-771, WX-780 and WX-781

The urokinase-type plasminogen activator (uPA), its cellular receptor (uPAR) and its inhibitor (PAl-1) are essential components of the plasmin activation cascade. Plasmin plays an important part in tissue remodeling and cell migration events and also in tumor-associated proteolytic activity due to activation of other enzymes, for example matrix metalloproteinases (MMPs). Thus, in breast cancer for example, high expression rates of these components support invasion, metastatic spreading and neoangiogenesis of tumors. The inhibition of tumor-associated proteolytic activity is therefore a novel concept for developing anti-tumor or anti-metastatic drugs for cancer patients.

Experiments were carried out in order to investigate the anti-tumor efficacy and anti-metastatic efficacy of WX-771, WX-780 and WX-781.

The assay system used was the BN-472 metastasizing breast tumor in brown Norwegian rats. The kind of tumor tissue used is described, for example, in Kort et al., J. Natl. Cancer Inst. 72 (1984) 709-713. The rats were purchased from Harlan Nederland, NL 5960-AD Horst, the Netherlands, and were from six to eight weeks old with a weight of from 128 g to 170 g.

### Solid Substances

### WX-340:

(4-Chlorobenzylsulfonyl-D-Ser-Gly-4-guanidinobenzyl-amide, hydrochloride salt) whose anti-tumor and anti-metastatic efficacy with subcutaneous injection had been disclosed was also studied for comparative purposes. For administration, 0.75 mg of WX-340 was dissolved in 20 ml of 5% (weight/volume) D-manitol in water and administered by subcutaneous injection in doses of 0.2 mg of drug/kg of rat.

### Control:

A phosphatidylcholine vehicle was administered as control and was obtained by introducing 400 mg of soya phosphatidylcholine in 20 ml of phosphate-buffered saline and 400 μl of absolute ethanol. 0.8 ml of the suspension was administered orally to the rats as control.

### WX-771:

WX-771 (7.5 mg) was introduced into 20 ml of a soya phosphatidylcholine solution as used also for the control. To administer 2 mg/kg, 0.8 ml of this suspension was administered orally. For a second group, the stock solution was diluted 1:10 to obtain a dose of 0.2 mg/kg when administering likewise 0.8 ml per rat.

### WX-780:

WX-780 (7.5 mg) was introduced into 20 ml of a soya phosphatidylcholine suspension according to the above-described control suspension. To administer a dose of 2 mg/kg, the rats were given 0.8 ml of said suspension. To administer a dose of 0.2 mg/kg, the stock solution was diluted 1:10, with 0.8 ml per rat still being administered.

### WX-781:

WX-781 (7.5 mg) was introduced into 20 ml of a soya phosphatidylcholine suspension according to the above-described control suspension. To administer a dose of 2 mg/kg, the rats were given 0.8 ml orally. To administer a dose of 0.2 mg/kg, the stock solution was diluted 1:10, with 0.8 ml/rat still being administered orally.

### Results:

The studies have demonstrated that WX-771, WX-780 and WX-781 exhibited significant anti-tumor and anti-metastatic action with oral administration of daily doses of 0.2 or 2 mg/kg for three weeks. The results are depicted in FIGS. 6 to 9.

TABLE 1
Synthesized compounds and calculated octanol/water
distribution coefficients (clogD)

| Formula | clogD | Name |
|---|---|---|
| 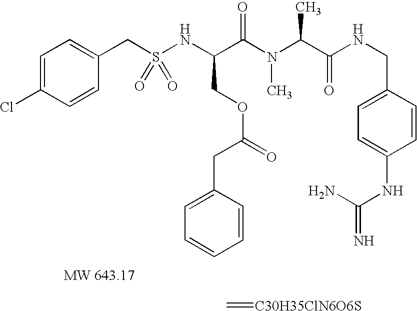 | 0.78 | WX-736 |
| 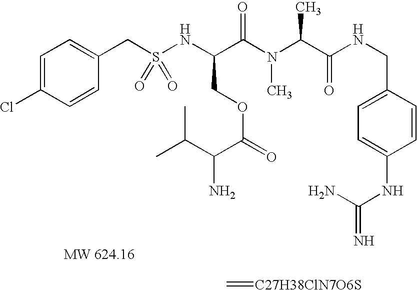 | −1.3 | WX-750 |
| 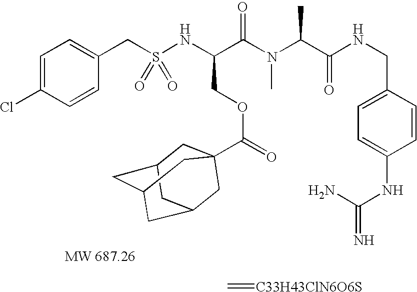 | 1.9 | WX-748 |
| 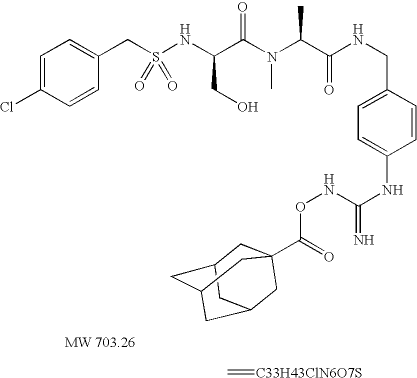 | 3.4 | WX-756 |
| 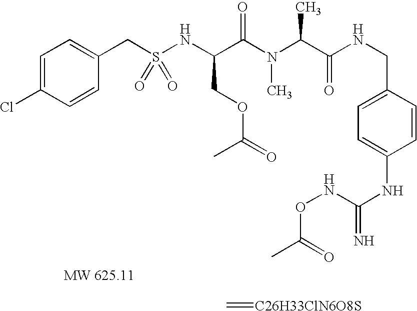 | 1.4 | WX-759 |
| 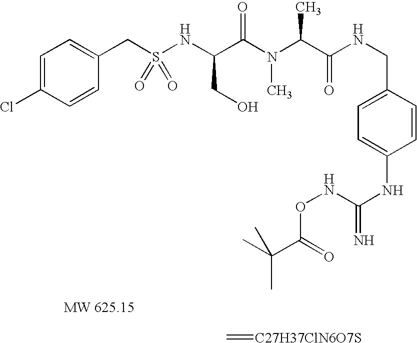 | n.d. | WX-760 |
| 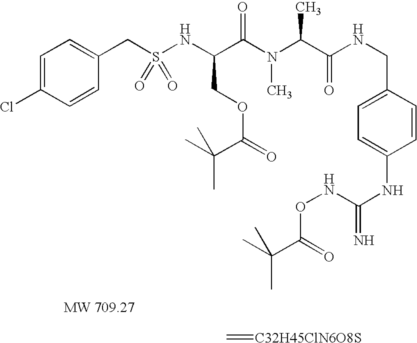 | 3.8 | WX-770 |
| 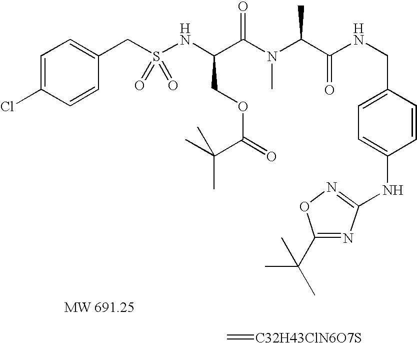 | n.d. | WX-780 |

n.d.: not determined

1. A medicament, which comprises, as an active compound, one or more compounds of the general formula I 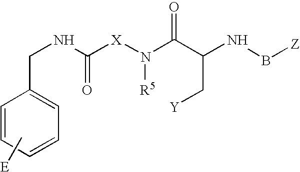
in which
E is a group from 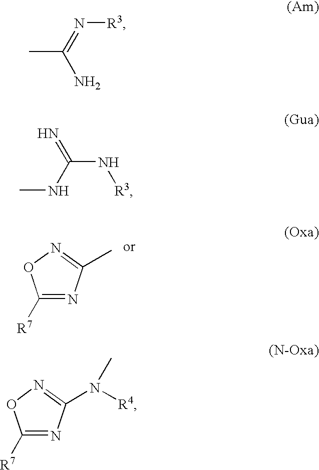
B is —SO₂— or —CO—,
X is —NR¹— or —CHR¹—,
Z is —R⁴, —OR⁴ or —NH—R⁴,
Y is —OR² or —NHR²,
R¹ is in each case independently —H, branched or straight-chain —C₁-C₆-alkyl, —C₂-C₆-alkenyl or —C₂-C₆-alkynyl, unsubstituted or substituted or a cyclic radical,
R² is —H, —R¹, —COR¹, —COOR¹ or —CON(R¹)₂,
R³ is H or —O—R⁸,
R⁸ is —H, —C₁-C₆-alkyl, —C₂-C₆-alkenyl or —C₂-C₆-alkynyl, unsubstituted or substituted, or —COR⁶ or —COOR⁶ or an oligo- or polyalkyleneoxy radical, for example with 2-50 —C₂-C₄-alkyleneoxy, for example ethyleneoxy, groups,
R⁴ is —H, —C₁-C₆-alkyl, —C₂-C₆-alkenyl or —C₂-C₆-alkynyl, unsubstituted or substituted, or a cyclic radical,
R⁵ is —H, —C₁-C₆-alkyl, —C₂-C₆-alkenyl or —C₂-C₆-alkynyl, unsubstituted or substituted, or a cyclic radical,
R⁶ is —H, branched or straight-chain —C₁-C₆-alkyl, C₂-C₆-alkenyl or —C₂-C₆-alkynyl, unsubstituted or substituted, or a cyclic radical,
R⁷ is H, branched or straight-chain, linear, mono-, bi- or polycyclic alkyl, alkenyl, alkynyl, carboxyalkyl, carboxyalkenyl, carboxyalkynyl, aryl, heteroaryl, carboxyaryl, carboxyalkylaryl, carboxyheteroaryl, —(CO)NR¹R⁴ or —COO—R⁴,
with each cyclic radical being able to carry one or more substituents, for example selected from the group consisting of —C₁-C₃-alkyl, —OR⁶ (e.g. —OH or —C₁-C₃-alkoxy), halogen, in particular Cl, ═O, —NO₂, —CN, —COOR⁶, —N(R⁶)₂, —NR⁶COR⁶, —NR⁶CON(R⁶)₂ and —OCOR⁶,
and it being possible for each alkyl, alkenyl or alkynyl to be straight-chain or branched and to carry one or more substituents, for example selected from the group consisting of halogen (F, Cl, Br, I), —OR⁶, —OCOR⁶, —N(R⁶)₂, —NR⁶COR⁶, COOR⁶, —NR⁶CON(R⁶)₂ or a cyclic radical,
it not being possible,
when Y=OH and E is Am or Gua,
for R³ or R⁸ to be H,
and when E=Am or Gua,
R³ is —O—R⁸ and R⁸ is —COR⁶ or —COOR⁶ or an oligo- or
or polyethylene oxide radical,
or R2 is —COR¹, —COOR¹ or —CON(R¹)₂,
or salts of said compounds and, where appropriate, pharmaceutically customary carriers, diluents or/and excipients.

2. The medicament as claimed in claim 1, characterized in that
it comprises one or more compounds of the general formula II 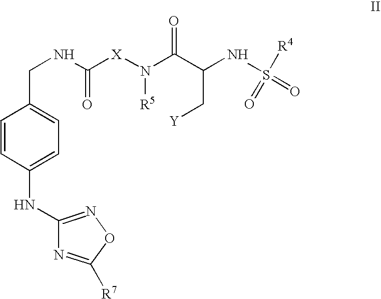
in which
X, Y, R⁴, R⁵ and R⁷ are as defined in claim 1,
or salts of said compounds.

3. The medicament as claimed in claim 1, in which
R⁴ is

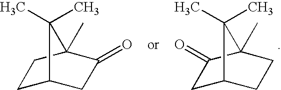

4. The medicament as claimed in claim 1, in which
R⁴ is a substituted or unsubstituted C₁-C₃-alkyl-aryl radical, in particular a benzyl radical, which may be unsubstituted or substituted with halogen or/and —NO₂ in the meta or para position, said halogen being selected from the group consisting of F, Cl, Br and I.

5. The medicament as claimed in claim 1, in which the compounds are selected from the group consisting of 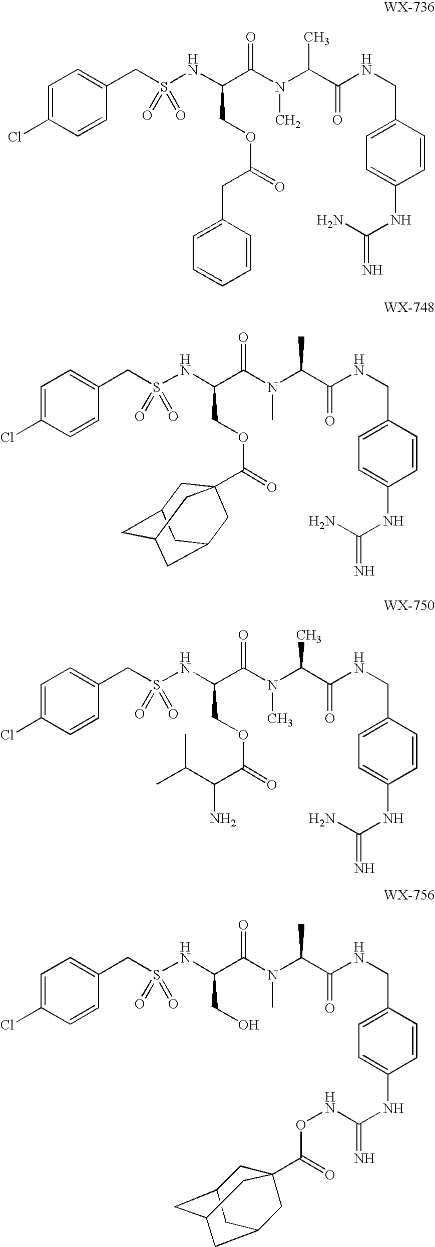 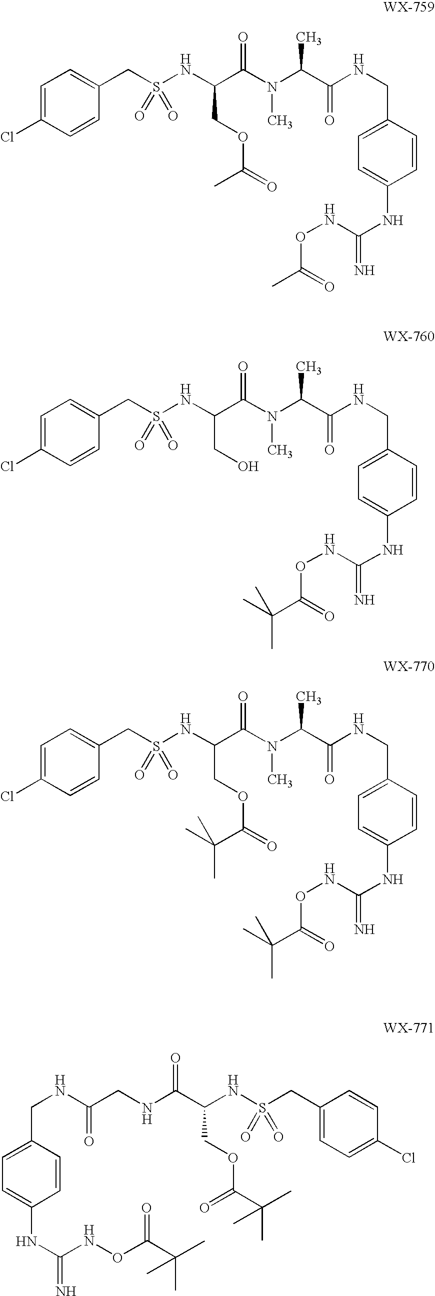 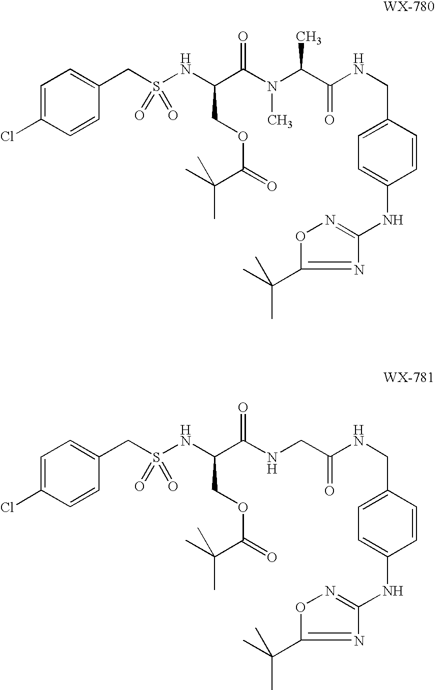
or salts thereof.

6. The medicament as claimed in claim 1, characterized in that
it is an orally administrable agent.

7. The use of a medicament as claimed in claim 1 for preparing a pharmaceutical composition for controlling diseases associated with pathological overexpression of urokinase and/or urokinase receptor.

8. The use as claimed in claim 7 for the treatment or prevention of tumors.

9. The use as claimed in claim 8 for the treatment or prevention of the formation of metastases.

10. The use as claimed in claim 9 for the treatment of primary tumors.

11. The use as claimed in claim 7, in which an orally administrable composition is prepared.

12. The use as claimed in claim 7, in which the composition is prepared in the form of tablets, coated tablets, capsules, pellets, solutions, emulsions or/and suspensions.

13. A compound of the formula I 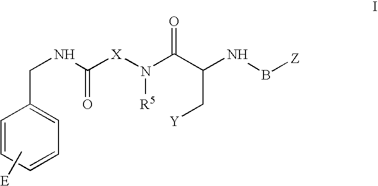
in which E, B, X, Z, Y and R⁵ are as defined in claim 1.

14. A compound of the formula II 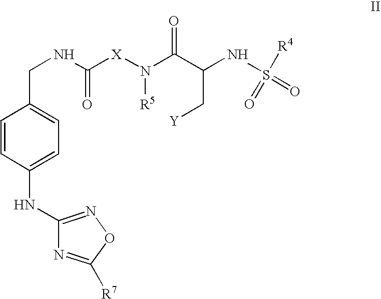
in which X, Y, R⁴, R⁵ and R⁷ are as defined in claim 1.

15. The compound as claimed in claim 13, selected from the group consisting of 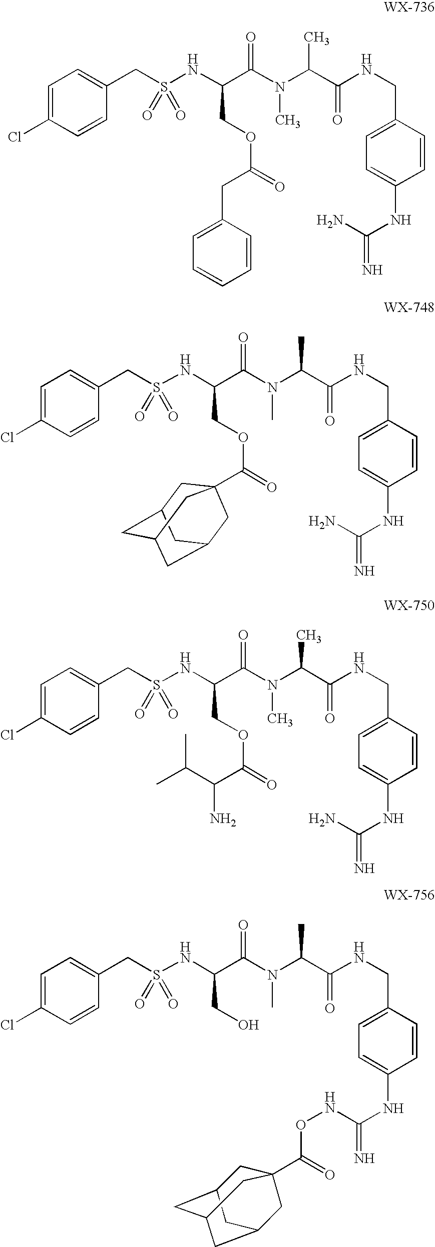 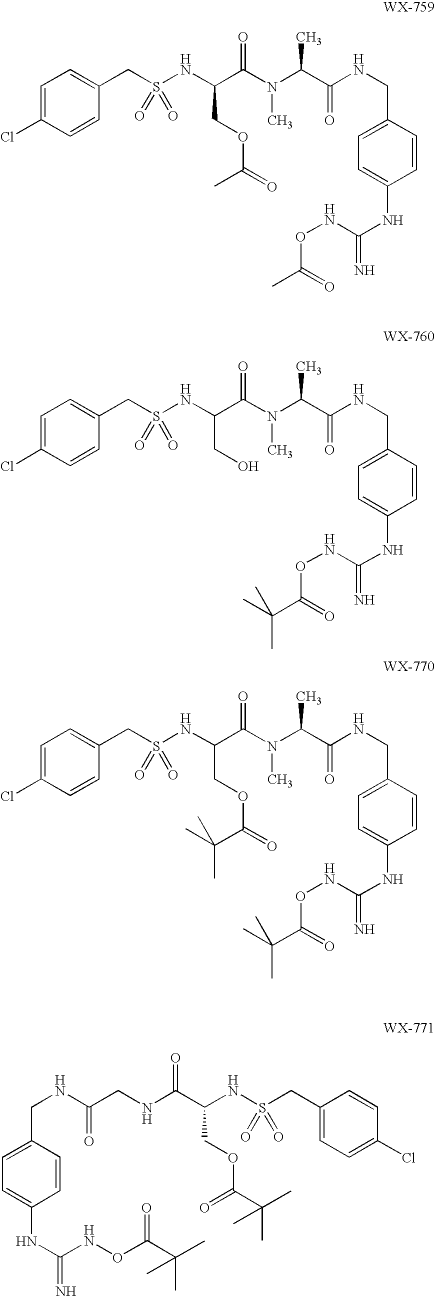 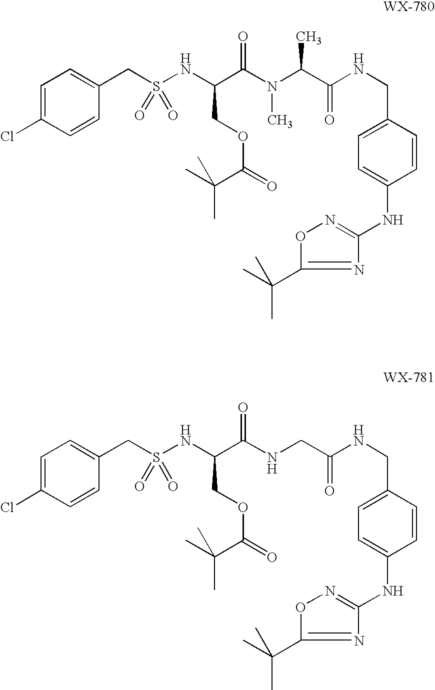
or salts thereof.

16. A process for inhibiting urokinase in living organisms by administering an active amount of at least one compound as claimed in claim 13.

17. A process for inhibiting urokinase in humans by administering an active amount of at least one compound as claimed in claim 13.

18. The use of the compound as claimed in claim 13 as a prodrug.
